(19) United States
(12) Patent Application Publication
(10) Pub. No.: US 2013/0149368 A1
(43) Pub. Date: Jun. 13, 2013

(54) BIOCELLULOSE DRESSING AND METHOD FOR PREPARING THE SAME

(71) Applicant: Chia Meei Food Industrial Corp., Taichung City (TW)

(72) Inventors: Chao-Ming Yu, Taichung City (TW); Chun-Chieh Lien, Taichung City (TW)

(73) Assignee: CHIA MEEI FOOD INDUSTRIAL CORP., Taichung City (TW)

(21) Appl. No.: 13/711,218

(22) Filed: Dec. 11, 2012

(30) Foreign Application Priority Data
Dec. 12, 2011 (TW) 100145788

(51) Int. Cl. A61L 15/28

(52) U.S. Cl. 424/445; 514/57; 427/2.31

(57) ABSTRACT

A biocellulose dressing contains a high content of a humectant with superior absorbency and promotes wound healing. The biocellulose dressing contains 10-20% (w/w) of water, 5-30% (w/w) of a microbial cellulose and 50-80% (w/w) of a humectant. A method for preparing dressing is also disclosed.

### CROSS-REFERENCE TO RELATED APPLICATION

This application claims priority of Taiwanese application no. 100145788, filed on Dec. 12, 2011.

### BACKGROUND OF THE INVENTION

1. Field of the Invention

This invention relates to a biocellulose dressing comprising water, microbial cellulose and a humectant. Also disclosed is a method for preparing the dressing.

2. Description of the Related Art

Wound healing process is affected by several factors, which include the size and type of wound (e.g., scald, trauma, surgery, contusion, etc.), the health and age of a patient and the drug that is being used. In general, the wound healing process includes three phases; (1) inflammatory phase; induction of an inflammatory response contributes to phagocytosis of bacteria and debris of necrotic tissue; (2) proliferative phase: events such as granulation tissue proliferation, angiogenesis, epithelialization, and contraction are key elements of making the wound smaller, thereby advancing the wound healing process to the next phase; and (3) maturation phase: collagen remodeling and capillary regression occur, thereby promoting complete healing of the wound.

Appropriate wound dressings are chosen in order to accelerate the wound healing process and reduce wound infection. Wound dressings can be categorized into the following types:
(1) Passive dressing (also known as traditional dressing): examples include gauze and tulle dressing; they are used to temporarily cover the wound, but they stick to the wound easily and requires frequent replacement;
(2) Interactive dressing: examples include film dressing, foam dressing, hydrocolloid dressing biocellulose dressing, etc.; they are usually transparent and moisture-vapor-permeable and oxygen-permeable and bacteria impermeable; and
(3) Bioactive dressing: examples include collagen dressing, alginate dressing, chitosan dressing, etc., these dressings can deliver active agents to the wound and promote healing thereof.

The biocellulose dressings are usually made of microbial cellulose. Microbial cellulose is derived from polysaccharide polymer made from cellulose-producing bacteria. The polysaccharide polymer has an ultrafine network structure that is mainly composed of D-glucopyranose units linked by β-1,4-glycosidic bonds, and has a degree of polymerization of about 2000 to 6000.

Commonly seen cellulose producing-microorganisms include: Sarcina sp., Pseudomonas sp., Rhizobium sp., Azotobacter sp., Aerobacter sp., Alcaligenes sp., Achromobacter sp., Agrobacterium sp., and Gluconacetobacter sp. (also known as Acetobacter sp., e.g., Gluconacetobacter xylinum is also known as Acetobacter xylinum).

Microbial cellulose possesses several properties that make it a good material for a wound dressing, including: (1) high hydrophilicity with water absorbent capacity of approximately 60-700 times its own weight; (2) difficult to break under tension due to its high tensile strength; and (3) difficult to break under compression due to its high wet strength.

Microbial cellulose dressings may be in the form of a film and have good strength and fluid handling ability (e.g., moisture absorption and donation). Because of their superior characteristics, such dressings are widely used in the medical industry and have bean used to treat various types of wounds, e.g., scald wound and chronic wounds.

U.S. Pat. No. 7,390,499 B2 discloses a microbial-derived cellulose dressing that can be used for the treatment of specific chronic wounds including pressure sores, venous and diabetic ulcers. The method for preparing the microbial-derived cellulose dressing includes: depyrogenating a microbial cellulose pellicle to provide a nonpyrogenic wound dressing; and adjusting the water content of the microbial cellulose dressing such that the wound dressing consists essentially of water and 1.5 to 4.3 wt % of microbial cellulose. In this method, the wound dressing is sot completely dried. The wound dressing can absorb fluid exudates in an amount of 20% to 200% bases on its weight. It addition, such dressing donates moisture in an amount greater than 75% based on its weight to a dry or necrotic portion at a chronic wound.

TW 200803924 (WO 2007/091801 A1) discloses a biocellulose sheet device for alleviating skin damage and relieving skin problem. The biocellulose sheet device comprises 1-50 wt % of microbial cellulose, 1-10 wt % of an active drug and 40-98 wt % of moisture. The biocellulose sheet device can further comprise 15-20 wt % of a water retention agent. In view of the Examples in WO 2007/091801 A1 publication, the water absorbency effect is attributed to the fibers of the microbial cellulose. There is no disclosure on as to how a water retention agent affects water absorbency.

### SUMMARY OF THE INVENTION

Therefore, the object of the present invention is to provide a biocellulose dressing and a method for preparing the same.

According to a first aspect, the present invention provide a biocellulose dressing, comprising 10-20% (w/w) of water, 5-30% (w/w) of microbial cellulose and 50-80% (w/w) of a humectant.

In a second aspect, the present invention provides a method for preparing a biocellulose dressing, comprising the steps of:
(a) providing a microbial cellulose pellicle including microbial cellulose and water;
(b) immersing the microbial cellulose pellicle into a humectant-containing solution to allow the microbial cellulose film to absorb a humectant of the humectant-containing solution until concentration of the humectant in the humectant-containing solution remains constant; and
(c) drying the microbial cellulose pellicle so as to obtain a biocellulose dressing having 10-20 wt % water.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 shows the shape and size of a biocellulose dressing according to the present invention that was used in a tensile test;

FIG. 2 is a plot showing the cumulative water vapor permeation value over the course of an eighty hour-water vapor permeation test;

FIG. 3 is a plot showing the cumulative wound healing rate along the various time points over the course of twenty days post-injury. The curves represent Sprague-Dawley (S.D.) rats from experimental and control groups 1, 2, and 3 in which the rats were applied with biocellulose dressing from the present invention, Skintemp, 3M Hydrocolloid and 3M Transparent, respectively;

FIG. 4 shows H&E staining of histological images obtained from wound tissues after 7 days of applying dressings to SD rats from experimental and control groups 1, 2 and 3 that were observed under 100× magnification, in which K indicates keratin layer, Epi indicates epidermis, N indicates neovascularization, and F indicates hair follicle; and

FIGS. 5A and 5B show H&E staining of histological images obtained from wound tissues after 14 days of applying dressings to SD rats from experimental and control groups 1, 2 and 3 under 100× (panel A) and 200× (panel B) magnifications, in which K indicates keratin layer, Epi indicates epidermis, N indicates neovascularization, and indicates hair follicle.

### DETAILED DESCRIPTION OF THE PREFERRED EMBODIMENTS

It is to be understood that, if any prior art publication is referred to herein,, such reference does not constitute an admission that the publication forms a part of the common general knowledge in the art, in Taiwan or any ether country.

For the purpose of this specification, it will be clearly understood that the word “comprising” means “including but not limited to ”, and that the word “comprises” has a corresponding meaning.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described.

The present invention provides a biocellulose wound dressing containing 10-20% (w/w) of water, 5-30% (w/w) of microbial cellulose and 50-85% (w/w) of a humectant.

Preferably, the biocellulose dressing contains 13-17% (w/w) of water, 8-15% (w/w) of microbial cellulose and 68-79% (w/w) of the humectant. In an example of this invention, the biocellulose dressing contains 14.92% (w/w) of water, 12.08% (w/w) of microbial cellulose and 73% (w/w) of the humectant.

Preferably, the biocellulose dressing has a thickness of 01-0.3 mm.

The present invention also provides a method for preparing the biocellulose dressing as described above, which includes the steps of:
(a) providing a microbial cellulose pellicle including microbial cellulose and water;
(b) immersing the microbial cellulose pellicle into a humectant-containing solution to allow the microbial cellulose film to absorb a humectant of the humectant-containing solution until concentration of the humectant in the humectant-containing solution remains constant; and
(c) drying the microbial cellulose pellicle so as to obtain a biocellulose dressing having 10-20 wt % water.

Given a commercially available biocellulose dressing having 0.54 wt % of the microbial cellulose, at lease 0.9 wt % of the humectants, based on the total weight of the immersed microbial cellulose pellicle obtained from step (b), must be presented in order to achieve at least a final concentration of 50% of the humectant in the biocellulose dressing.

According to the present invention, the microbial cellulose pellicle is made using cellulose-producing bacteria. The cellulose-producing bacteria include those that may be easily obtained by one having ordinary skill an the art (e.g., commercially available from domestic or foreign depository institutions), or those isolated and purified from natural resources by methods known to a skilled artisan.

Examples of the cellulose-generating microorganisms include, but are not limited to, Gluconacetobacter sp. (also known as Acetobacter sp.), Sarcina sp., Pseudomonas sp., Rhizobium sp., Azotobacter sp, Aerobacter sp., Alcaligenes sp., Achromobacter sp., Agrobacterium sp and combinations thereof.

According to the present invention, the microbial cellulose pellicle can be a commercial product, e.g., Nata (Chia Meei Vietnam Food Industrial Corporation), high fiber Nata (Hainan Yeguo Foods Co., Ltd.), biocellulose (Hainan Yide Food Co, Ltd., catalog number: N200789173034) and biocellulose substrate (Limmer Biotech Corp.). In an example of the present invention, the microbial cellulose pellicle is Nata from Chia Meei Vietnam Food Industrial Corporation, which is a microbial cellulose pellicle obtained from Gluconacetobacter xylinum.

According to the present invention, the microbial cellulose pellicle is subjected to a dehydration step before immersing in the humectant-containing solution such that the biocellulose dressing contains 85-95% (w/w) water and 5-15% (w/w) microbial cellulose.

According to the present invention, dehydration of the biocellulose dressing and the microbial cellulose pellicle can be processed using a known technique including, but not limited to, air drying, shade drying, vacuum drying and freeze drying.

The humectant is a hydrophilic material. Preferably, the hydrophilic humectant can be polyol, saccharide and cellulose derivative. Examples of the hydrophilic humectant include glycerol, lactose, sodium carboxymethylcellulose, sucrose, glucose, sodium methylcellulose, glucitol, starch, dextrin, and combinations thereof. In an example of this invention, the humectant is composed of glycerol, lactose and sodium carboxymethylcellulose.

The microbial cellulose pellicle contains 5% (w/w) of glycerol, 1% (w/w) lactose and 0.05% (w/w) of sodium carboxymethylcellulose after immersing in humectant-containing solution.

The content of the microbial cellulose, water and humectant can vary depending upon the method of preparation and the ultimate end use of the biocellulose dressing. According to the present invention, the preparation methods for the biocellulose dressing can be modified according to the desired water content, microbial cellulose and concentration of the humectant. Such modification includes adjusting the concentration of the humectant-containing solution, the duration of immersing in the humectant-containing solution, drying conditions, etc.

The biocellulose dressing of this invention can be sterilized using commonly used techniques which include, but are not limited to, gamma-ray sterilization, electron beam sterilization, heat sterilization and high-pressure sterilization. Gamma-ray sterilization is used in an example of this invention.

The biocellulose dressing may incorporate active agents to promote wound closure and/or prevent microbial infections. The suitable active agents include, but are not limited to, collagen, alginate, anti-inflammatory agents (e.g., corticosteroids), anti-bacterial agents (e.g., chitosan, nano silver and nisin, etc.) and wound-healing agents (e.g., epidermal growth factor (EGF), acidic fibroblast growth factor, vascular endothelial growth factor (VEGF), platelet-derived growth factor (PDGF), insulin-like growth factor (IGF), transforming growth factor (TGF), growth and differentiation factor (GDF), bone morphogenetic protein (BMP), demineralized bone matrix (DBM), factor VIII and sulfacetamide, etc.). The choice of the active agent and the dosage thereof depend on the purpose of the end use.

The present invention also provides a method for healing a wound, which includes application of the biocellulose dressing of this invention to a wound.

The duration of the application of the biocellulose dressing and the frequency of replacement are adjusted according to the following factors: the type, location, size, depth, and severity of the wound; the amount of wound exudate, well as the degree of healing. In general, the biocellulose dressing of the present invention should be replaced every two to five days.

### EXAMPLES

### Preparation of the Biocellulose Dressing

A commercially available microbial cellulose pellicle (Nata, Chia Meei Vietnam Food Industrial Corporation) was purified before further experimentation. The microbial cellulose pellicle having a thickness of approximately 2-10 mm was soaked in 0.25 wt % NaOh at 25° C. for 8 hours followed by immersing in 0.2 wt % H₂O₂ at 25° C. for 6 hours. The microbial cellulose pellicle was washed several times with water to remove bacteria cells and chemicals therein. Subsequently, the washed microbial cellulose pellicle was sterilized at 95° C. for 10 minutes, and was compressed using a compressor (PX1, Nan Kong Machinery CO., LTD.) under a 5 kg/cm² pressure for 60 seconds to compress some of the moisture. The compressed microbial cellulose pellicle had a thickness of 0.2-1 mm measured by a dial thickness gauge (Peacock, MODEL G). The composition of the microbial cellulose pellicle was determined and assessed using conventional techniques. The resultant microbial cellulose pellicle had a water content of 85-95% (w/w) and 5-15% (w/w) of microbial cellulose.

Thereafter, the microbial cellulose pellicle was immersed in a humectant-containing solution in order to incorporate the hydropholic humectants (i.e., glycerol, lactose and sodium carboxymethyl cellulose) of the humectant-containing solution in the microbial cellulose pellicle. The humectant-containing solution contained 6.3 wt % of glycerol, 1.3 wt % of lactose and 0.062 wt % of sodium carboxymethyl cellulose in water. The ratio of the microbial cellulose pellicle to the humectant-containing solution was 1:4 (w/w) and the immersion time was 24 hours. Such immersion allows the microbial cellulose pellicle to contain the humectant mainly by solvent exchange, thus reaching a final concentration of 5% (w/w) glycerol, 1% (w/w) of lactose and 0.06% (w/w) of carboxymethyl cellulose sodium in the microbial cellulose pellicle. Thereafter, the microbial cellulose pellicle underwent shade drying for 96 hours to obtain a biocellulose dressing. The biocellulose dressing had a thickness of approximately 0.1-0.3 mm which was measured using a dial thickness gauge.

The content of the biocellulose dressing was determined by Environmental Science and Technology Research Center at Yuan Ze University, Taiwan, in accordance with techniques known to one having ordinary skill in the art. The resultant biocellulose dressing had 14.92% (w/w) of water, 12.08% (w/w) of microbial cellulose and 73% (w/w) of the humectant. The resultant biocellulose dressing was tailored into desired sizes for the following experiments.

### Analysis of the Physical Properties of the Biocellulose Dressing

### A. Tensile Test

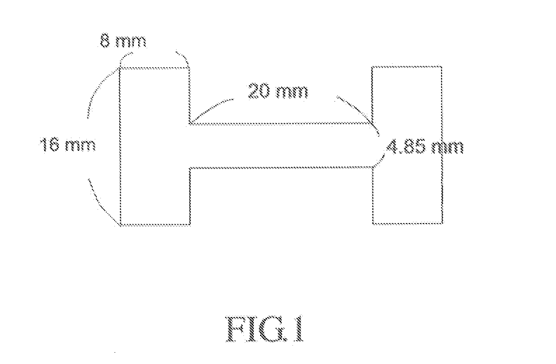

The aforesaid biocellulose dressing (the shape and size thereof are shown in FIG. 1; was subjected to a tensile test using a tensile testing machine (HT-8504, Hung TA Instrument Co., Ltd.) operated in accordance with the manufacturer's instructions. The Young's modulus, fracture strength and elongation were determined at a pulling speed of 100 mm/min. Data are shown as mean±standard deviation (SD) from five repeated measurements of five biocellulose dressings,

### B. Water Vapor Permeation Test

The aforesaid biocellulose dressing (4 cm×4 cm) was used to cover an opening of a 50 mL centrifuge tube containing 35 mL of water. The covered centrifuge tube was weighed and allowed to stand at 37° C. for 78 hours. At various designated time points, specifically, 0, 1, 2, 3, 4, 5, 6, 7, 23, 24, 27, 30, 48, 51, 54, 73, 75 and 78 hour, the weight of the covered centrifuge tube was measured. The water vapor permeation value was determined using the following equation (1):

A=B−C equation b 1)

wherein, A=water vapor permeation value (g)
B=initial weight of the covered centrifuge tube measured at 0 hour (g)
C=the weight of the covered centrifuge tube measured at the designated time point (g)

Data are shown as mean±standard deviation from five repeated measurements of five sheets of biocellulose dressings.

### C. Water Absorption Measurement

The aforesaid biocellulose dressing (4 cm×4 cm) was weighed, followed by immersing in water (dH2O) for 2 hours. At various time points during the period of immersion, specifically, 10, 30, 60 and 120 minutes, the biocellulose dressing was removed from the water and weighed. The water absorption rate was determined using the following equation (2):

D=(F/E)×100% equation (2)

wherein, D=water absorption rate (%)
E=weight before immersing in water (g)
F=weight after immersing in water at various time points (g)

Data are shown as the average of ten measurements from three sheets of biocellulose dressing.

### COMPARATIVE EXAMPLE

A bio-cellulose sheet according to TW 200803924 (WO 2007/091801) was used as a comparative example for a side by side comparison to those obtained in Example 1 of the present invention. To be specific, gel-phase microbial cellulose obtained in Examples 1-2 of TW 200803924 (WO 2007/091801) was compressed in an air compressor into a thickness of 0.4-0.8 mm, thus providing a wet sheet of microbial cellulose with a moisture content of 80%. Thereafter, the microbial cellulose was immersed in 50% mineral oil in a 1:5 w/w ratio for 24 hours. Excess water drops on the microbial cellulose were wiped off after being taken out of the mineral oil and subjected to water absorption measurements as described below. Data obtained from the microbial cellulose from TW 200803924 (WO 2007/091801) are shown as the mean of three repeated measurements from three sheets of biocellulose dressing.

### Result

### A. Tensile Test

Young's modulus, fracture strength and elongation of the biocellulose dressing were 33.57±4.13 MPa, 14.77±2.05 MPa, and 32.17±2.85%, respectively. These results reveal that the biocellulose dressing of the present invention has good elasticity and ductility, which suggest that the biocellulose dressing can be appressed and securely attached to the wound, thereby eliminating the possibility of displacement of the dressing and irritation of the wound.

### B. Water Vapor Permeation Test

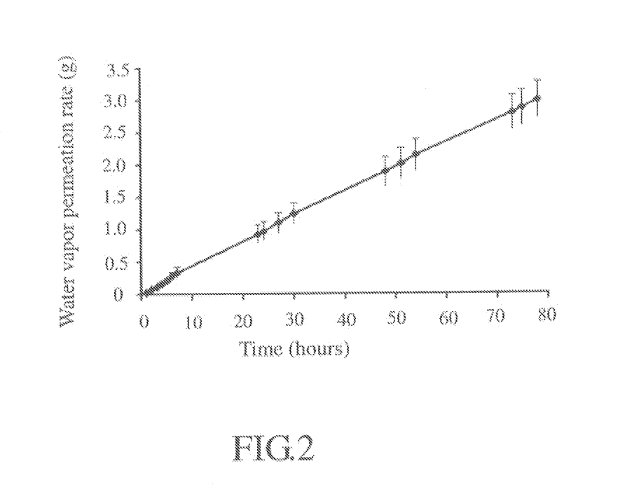

FIG. 2 a is a plot of water vapor permeability by showing the cumulative water loss (weight change of the microbial cellulose film) during the course of 80 hours at different time points. The results suggest that the water in the centrifuge tube evaporates at a constant rate and permeates through the biocellulose dressing that is covered on top of the open centrifuge tube. These results suggest that the biocellulose dressing of the present invention has good permeability.

### C. Water Absorption Measurement

Water absorbency of the dressing of the current invention and the bio-cellulose sheet from Example 1 of TW 200803924 are shown in Table 1. As shown in Table 1, water absorbency of the bio-cellulose sheet from TW 200803924 is approximately 100%, and does not significantly increase over time. In contrast, water absorbency of the biocellulose dressing of the present invention is approximately 800% after 10 minutes of immersion, and is stably increased over the 120 minute-testing period. These results show that the water absorbency of the biocellulose dressing of the present invention has good absorbency, and suggest that the biocellulose dressing of the present invention can promote wound healing by absorbing large amounts of wound exudates.

TABLE 1

Water absorbancy (%)

| | Biocellulose | Bio-cellulose |
|---|---|---|
| Time | dressing of the | sheet from |
| (minutes) | present invention | TW 200803924 |
| 10 | 797 | 105 |
| 30 | 1138 | 107 |
| 60 | 1357 | 108 |
| 120 | 1541 | 110 |

The biocellulose dressing is air-dried which allows the dressing to contain residual moisture, yet the high content of humectant renders the biocellulose dressing of the present invention able to absorb large amount of exudates. Such properties allow the biocellulose dressing to be replaced less frequently and avoid the disturbance of the wound.

### Assessment of Wound Healing of the Biocellulose Dressing In Animal Models

### <Materials And Methods>

### 1. Animals

Male Sprague-Dawley (SD) rats (approximately 200 g, 8 weeks old purchased from BioLasco Taiwan Co., Ltd.) were used in the following experiments. All animals were housed in a 12-hour light/dark cycle, with constant room temperature (22° C.) and controlled relative humidity (42%), food and water ad libitum. Prior to the experiment, the animals were given an acclimation period of at least two weeks. Feeding, management and handling of all experimental procedures were in accordance with the National Institute of Health (NIH) Guide for the Care and Use of Laboratory Animals.

### 2. Sterilization of the Biocellulose Dressing

Sterilization of the biocellulose dressing prepared in the section of “Preparation of the biocellulose dressing” (4 cm×4 cm) was subjected to gamma-ray (γ-ray) irradiation (dose of 40 kGy). The sterilized biocellulose dressing was used in the following experiments.

### 3. Skin Wound Formation

The dorsal part of the SD rats was shaved and disinfected with iodine (tincture of iodine) and 70% alcohol. Thereafter, five wounds (approximately 1.2 cm×1.2 cm with a depth of 2-3 mm) were created using a scalpel on the dorsal paravertebral skin of the SD rats.

### 4. Application of the Biocellulose Dressing

SD rats were randomly divided into an experimental group and three control groups (i.e., control groups 1, 2 and 3) (n=9/group), wherein wounds of the SD rats in each group were created according to the aforementioned Item 3. The sterilized biocellulose dressing obtained in the aforementioned Item 2, was applied to the wounds of the SD rats in the experimental group. The wounds on the SD rats from control group 1, 2 nod 3 were covered with other commercially available wound dressings, i.e., SkinTemp collagen dressing (purchased from BioCore, hereinafter referred to as SkinTemp), Tegaderm™ Hydrocolloid Dressing (purchased from 3M, hereinafter referred to as 3M Hydrocolloid) and Tegaderm™ transparent dressing (purchased from 3M, hereinafter referred to as 3M Transparent), respectively. The dressings were replaced every two to three days over a course of a twenty day-experimental period while the rats were under inhalational anesthesia using isoflurane.

### 5. Data Analysis

The rats from experimental group and three control groups were subjected to the following analysis, which includes wound healing rate and histological examination.

Wound healing rate was determined by the histological images of the wound taken prior to and 1, 3, 6, 8, 10, 13, 15. 17 and 20 days after application of the dressings and were analyzed according to the method described in the following section A.

Histological analyses of the wound tissue samples were collected from three rats per group by sacrificing at 7, 14 and 21 days after dressing application. The wound tissues obtained were analyzed according to the method described in the following section B.

### Assessment of Wound Healing Efficiency For Different Wound Dressings

### A. Determination of Wound Healing Rate

The area of the skin wound of the SD rats in each group was assessed by analyzing the image taken at each designated time point using ImageJ software (NIH). The rate of wound healing was determined by the following equation (3):

G=((H−I)/H)×100% equation (3)

wherein, G=the rate of wound healing,

H=the area of the wound before using the dressing,
I=the area of the wound after using the dressing.
Data are shown as mean±standard deviation.

### B. Histopathological Examination

The wound tissue samples obtained at room temperature were fixed for at least 24 hours in 4% paraformaldehyde at room temperature. Thereafter, samples were processed through graded ethanol solutions and embedded in paraffin blocks using standard protocols. 5 μm sections were obtained and subjected to hematoxylin-eosin staining. The stained samples were analyzed under an optical microscope (Eclipse 80i, Nikon) with 100× and 200× magnifications.

### Results

### A. Determination of Wound Healing Rate

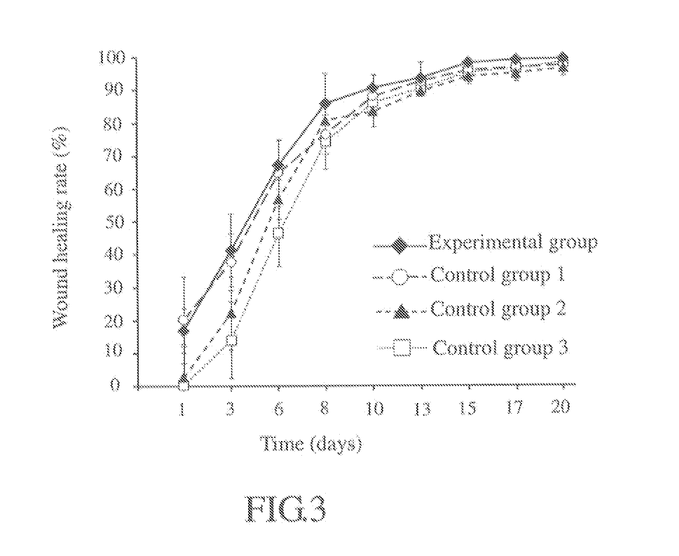

FIG. 3 shows the wound healing rate of the SD rats from either the experimental or control groups over the course of twenty days. As shown in FIG. 3, the rate of wound healing of the SD rats either in the experimental group and the control group 1 (Skintemp) were higher than that in control groups 2 (3M Hydrocolloid) and 3 (3M Transparent) after 6 days of applying the wound dressing, and had reached more than 60% of recovery. By the eighth day, the recovery of the wound from the experimental group had reached approximately 85%. By the twentieth day, the healing of the wound is near completion. These results suggest that the wound healing effect of the biocellulose dressing of the present invention is similar or even superior to commercially available wound dressings.

### B. Histological Examination

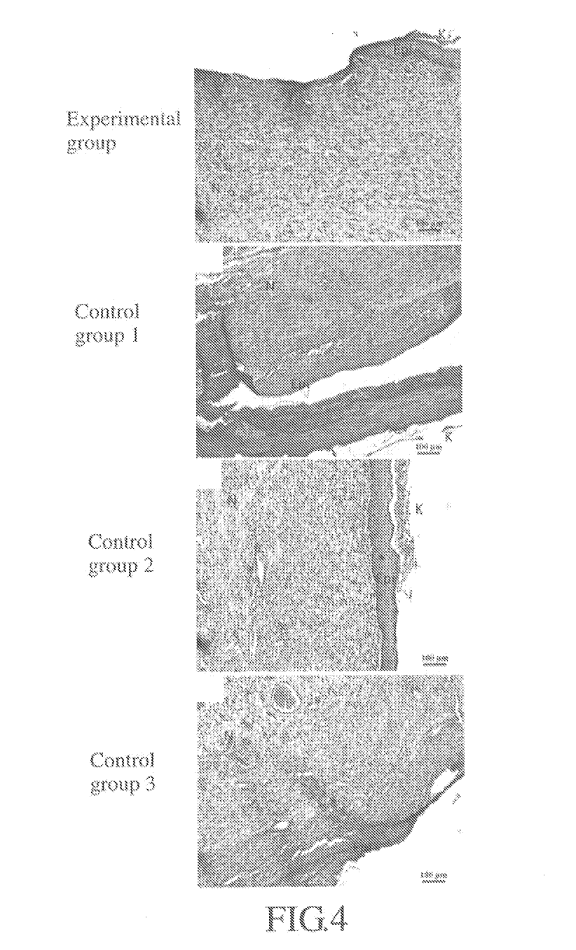

FIG. 4 shows representative histological images of wound tissues obtained after seven days of applying the dressing to the wound of the SD rats from each group under a magnification of 100×. As shown in FIG. 4, granulation tissue proliferation and angiogenesis were observed in the wound of SD rats in each group after 7 days of application of the dressing. In the experimental group, the wound is covered with superficial neoepithelium (SN) (which leads to formation of the epidermis, labeled “Epi”, in FIG. 4). During the healing process, SN proliferates to repair the wounded epidermis. Neovascularization (labeled “N” in FIG. 4), proliferation and migration of the epithelium were also observed.

In control group 1, the three dimensional structure of the SkinTemp collagen dressing can act as an extracellular matrix, allowing the cells to enter collagen reticulation of the SkinTemp collagen dressing. This leads to the migration of epithelial cells, and further promotes wound healing. Upon completion of epithelialization, the dressing was peeled off together with the outer layer of keratin.

In control group 2 (3M hydrocolloid), epithelial cell migration and epithelialization were present in addition to neovascularization, together with the formation of a small number of hair follicle (labeled “F” in FIG. 4).

In control group 3 (3M transparent, the migration of epithelial cells and epithelialization of the wound were not obvious when compared to other groups, and the wound healing process was relatively slow.

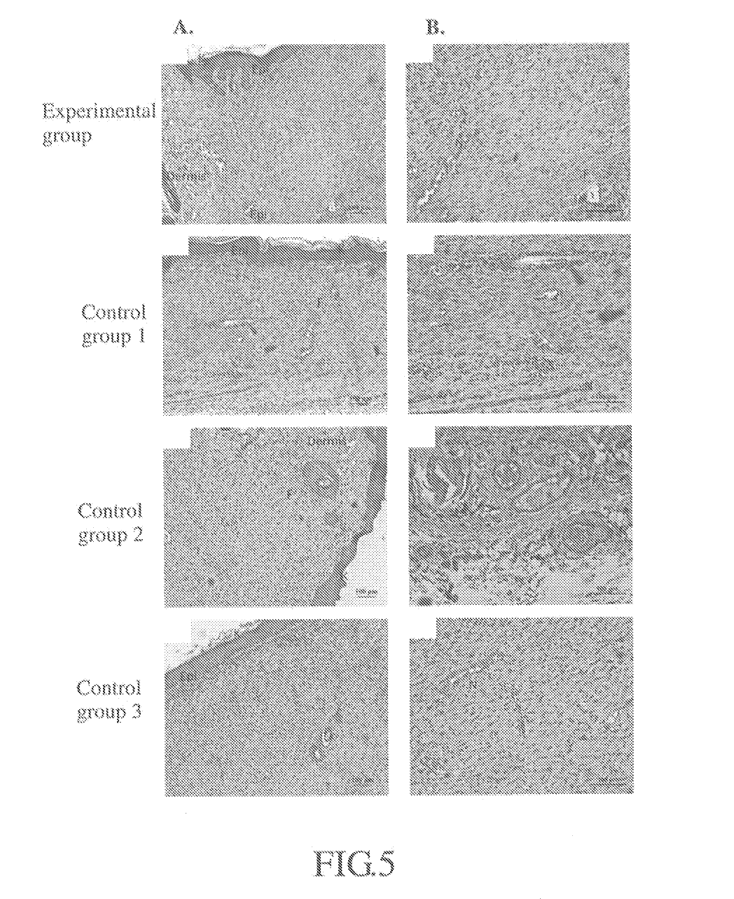

FIGS. 5A and 5B show representative histological images of wound tissues obtained after fourteen days of applying the dressing to the SD rats from each group under 100× and 200× magnifications, respectively. As shown in FIGS. 5A and 5B, neoepithelium and stratum corneum were clearly observed, and collagen remodeling was presented. Specifically, in experimental group, proliferation of the dermal tissue (labeled “Dermis” in FIG. 5) was observed at the edge of the wound. Distinct layers of neoepithelium and keratin were formed in the wound bed. In control group 1, the wound showed a thicker keratin layer (labeled “K” in FIG. 5), hair follicle formation and regression of excess capillaries formed during neovascularization. In control group 2 (3M Hydrocolloid), dermal tissue was observed at the edge of the wound (labeled “Dermis” in FIG. 5), with complete keratin layer and hair follicle formation. In control group 3 (3M Transparent), neovascularization was obvious, but hair follicle formation was not observed.

These results indicate the biocellulose dressing of the present invention can promote granulation tissue proliferation and epithelialization. The effect of wound healing promoted by the biocellulose dressing of the present invention is similar to or superior to those dressings that are commercially available.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A biocellulose dressing, comprising 10-20% (w/w) of water, 5-30% (w/w) of microbial cellulose and 50-80% (w/w) of a humectant.

2. The biocellulose dressing according to claim 1, prepared by a process including the following steps:
(a) providing a microbial cellulose pellicle including microbial cellulose and water;
(b) immersing said microbial cellulose pellicle into a humectant-containing solution to allow the microbial cellulose pellicle to absorb a humectant of the humectant-containing solution until concentration of said humectants in said humectant-containing solution remains constant; and
(c) drying said microbial cellulose pellicle so as to obtain a biocellulose dressing having 10-20 wt % water.

3. The biocellulose dressing according to claim 2, wherein step (c) is conducted by techniques selected from the group consisting of air drying, shade drying, vacuum drying and freeze drying.

4. The biocellulose dressing according to claim 2, wherein said humectant is a hydrophilic material.

5. The biocellulose dressing according to claim 4, wherein said humectant is selected from the group consisting of glycerol, lactose, sodium carboxymethylcellulose, sucrose, glucose, sodium methylcellulose, glucitol, starch, dextrin, and combinations thereof.

6. The biocellulose dressing according to claim 5, wherein said humectant is composed of glycerol, lactose and sodium carboxymethylcellulose.

7. The biocellulose dressing according to claim 6, wherein, after step (b), said microbial cellulose pellicle contains 5% (w/w) glycerol, 1% (w/w) lactose and 0.06% (w/w) sodium carboxymethylcellulose.

8. The biocellulose dressing according to claim 2, wherein, in step (a), said microbial cellulose pellicle is produced by microorganisms selected from the group consisting of Gluconacetobacter sp, Sarcina sp., Pseudomonas sp., Rhizobium sp., Azotobacter sp., Aerobacter sp., Alcaligenes sp., Achromobacter sp., Agrobacterium sp. and combinations thereof.

9. The biocellulose dressing according to claim 2, wherein the process further includes, before step (b), dehydrating said microbial cellulose pellicle such that said biocellulose dressing contains 85-95% (w/w) water and 5-15% (w/w) microbial cellulose.

10. The biocellulose dressing according to claim 2, wherein the process further includes, sterilizing said biocellulose dressing by gamma-ray sterilization, electron beam sterilization, heat sterilization or high-pressure sterilization.

11. The biocellulose dressing according to claim 1, comprising 13-17% (w/w) of water, 8-15% (w/w) of said microbial cellulose and 68-79% (w/w) of said humectant.

12. The biocellulose dressing according to claim 1, wherein said biocellulose dressing has a thickness of 0.1-0.3 mm.

13. A method for preparing a biocellulose dressing comprising the steps of:
(a) providing a microbial cellulose pellicle including microbial cellulose and water;
(b) immersing said microbial cellulose pellicle into a humectant-containing solution to allow the microbial cellulose pellicle to absorb a humectant of the humectant-containing solution until concentration of said humectant in said humectant-containing solution remains constant; and
(c) drying said microbial cellulose pellicle so as to obtain a biocellulose dressing having 10-20 wt % water.

14. The biocellulose dressing according to claim 13, wherein said humectant is a hydrophilic material.

15. The method according to claim 14, wherein said humectant is selected from the group consisting of glycerol, lactose, sodium carboxymethylcellulose, sucrose, glucose, sodium methylcellulose, glucitol, starch, dextrin, and combinations thereof.

16. The method according to claim 15, wherein the humectant is composed of glycerol, lactose and sodium carboxymethylcellulose.

17. The method according to claim 16, wherein, after step (b), said microbial cellulose pellicle contains 5% (w/w) glycerol, 1% (w/w) lactose and 0.06% (w/w) sodium carboxymethylcellulose.

18. The method according to claim 13, wherein said biocellulose dressing is produced by microorganisms selected from the group consisting of Gluconacetobacter sp, Sarcina sp., Pseudomonas sp., Rhizobium sp., Azotobacter sp., Aerobacter sp., Alcaligenes sp., Achromobacter sp., Agrobacterium sp. and combinations thereof.

19. The method according to claim 13, further comprising, before step (b), dehydrating said microbial cellulose pellicle such that said biocellulose dressing contains 85-95% (w/w) water and 5-15% (w/w) microbial cellulose.

20. The method according to claim 13, further comprising, after step (c), a step of sterilizing the biocellulose dressing by gamma-ray sterilization, electron beam sterilization, heat sterilization high-pressure sterilization.

21. The method according to claim 13, wherein said biocellulose dressing comprises 10-20% (w/w) of water, 5-30% (w/w) of the microbial cellulose and 50-80% (w/w) of the humectant.

22. The method according to claim 13, wherein said biocellulose dressing comprises 13-17% (w/w) of water, 8-15 % (w/w) of the microbial cellulose and 68-79% (w/w) of the humectant.

23. The method according to claim 13, wherein the biocellulose dressing has a thickness of 0.1-0.3 mm.
